(19) United States
(12) Patent Application Publication
(10) Pub. No.: US 2005/0136114 A1
(43) Pub. Date: Jun. 23, 2005

(54) Taste masked pharmaceutical compositions comprising bitter drug and pH sensitive polymer

(75) Inventors: Mohan G. Kulkarni, Maharashtra (IN); Anupa R. Menjoge, Maharashtra (IN)

Correspondence Address: NIXON & VANDERHYE, PC, 1100 N GLEBE ROAD, 8TH FLOOR, ARLINGTON, VA, 22201-4714, US

(73) Assignee: COUNCIL OF SCIENTIFIC AND INDUSTRIAL RESEARCH, New Delhi (IN)

(21) Appl. No.: 10/739,409

(22) Filed: Dec. 19, 2003

(51) Int. Cl. A61K 9/20; A61K 9/14

(52) U.S. Cl. 424/486

(57) ABSTRACT

The present invention discloses pharmaceutical compositions comprising of pH sensitive polymers used for taste masking highly bitter drugs. The pH sensitive polymer acts as a reverse enteric coating, which is soluble in the acidic pH range 1.0 to 3.0 normally found in the stomach but is insoluble in the pH range 3.5 to 7 thus inhibiting the release of the bitter drug at the pH of saliva and also at the pH of reconstitution medium in case of liquid orals.

### FIELD OF INVENTION

The present invention relates to taste masked compositions comprising a bitter drug and a pH sensitive polymer and methods for preparing the same. The present invention also relates to a process for the preparation of a taste masked pharmaceutical composition comprising bitter drug and a pH sensitive polymer.

### BACKGROUND OF INVENTION

Although a variety of delivery systems are being developed for different routes of administration like the oral, parenteral, nasal and transdermal, the oral route remains attractive for drug delivery because this mode of administration is an easy, convenient, noninvasive and familiar method of drug delivery. The majority of prescribed drugs are designed for oral application since they can be self-administered by the patient without hospitalization. Oral dosage forms are designed according to the nature of the drug, the nature of application and the need for any special effects. The common oral dosage forms include: liquid mixtures like solutions, suspensions, solid dosage forms like tablets and capsules and liquid filled capsules etc. The solid dosage forms are further modified depending on the therapeutic action desired, like controlled, extended or delayed release. However, patients at the extremes of age, such as children and the elderly, often experience difficulty in swallowing solid oral dosages forms. For these patients the drugs are mostly provided in liquid dosage forms such as solutions, emulsions and suspensions. These dosage forms usually lead to perceptible exposure of the active drug ingredient to the taste buds, which is a very serious problem when the drug has an extremely unpleasant or bitter taste.

The bitter taste of the drugs, which are orally administered, is disadvantageous in several aspects. Taste is an important parameter governing the compliance. The disagreeable taste of drugs causes difficulties in swallowing or causes patients to avoid their medication thereby resulting in low compliance of patients. Conventional taste masking techniques such as use of sweeteners, amino acids, flavoring agents are often unsuccessful in masking the taste of the highly bitter drugs like quinine, barberin, etoricoxib, antibiotics like levofloxacin, ofloxacin, sparfloxacin, ciprofloxacin, cefuroxime axetil, erythromycin and clarithromycin. Thus taste-masking technologies are considered important and developed by many researchers.

Taste masking is a major problem when the drugs are extremely unpleasant and bitter and this problem is not restricted to the liquid oral compositions like solutions, dry syrup and suspensions but may also be encountered during the formulation of chewable tablets or dispersible tablets wherein these dosage forms usually lead to perceptible exposure of active ingredient to taste buds. Depending on the type of dosage form, various methods have been employed to overcome the unpleasant taste and bitterness of the drug.

Patent Application WO 03/13470 discloses the use of ammonium glycyrrhyzinate to taste-mask the formulation comprising of a dry blend of paroxetine and a glycyrrhyzinate formulated as a dispersible powder or moulded into a dispersible or chewable tablet. Patent Application WO 03/11227 discloses a taste masked composition for the delivery of ibuprofen which causes a throat catch in the form of chewable ibuprofen tablets with the polymer, carbomer 934. European Patent EP 1219291 discloses chewable tablets and texture masked particles of the active ingredient, acetaminophen which is coated by a taste masking polymer ethyl cellulose and a film forming polymer and a texture masking coating solution of hydroxypropyl methyl cellulose and polyethylene glycol 800 and acesulfame potassium.

In yet another patent application JP 2002363066 the taste masked pharmaceutical or food composition is disclosed which is suitable for formulation as granule, tablet or a chewable tablet. The taste masked fine granule is obtained by using polymers such as ethyl cellulose, hydroxy propyl cellulose. European patent EP 1166777 discloses yet another chewable tablet made from taste masked particles. The active ingredient ibuprofen was coated by the enteric polymer HPMCP and an insoluble film forming agent cellulose acetate and chewable tablets with no throat burn were prepared from the coated particles by blending with aspartame, acesulfame potassium, citric acid, granular mannitol, fumaric acid, microcrystalline cellulose, and flavor.

Taste masking techniques are extended to the dispersible dosage forms and rapidly disintegrating tablets, too. The patent application WO 01/58449 discloses the water dispersible powder and tablets of paroxetine for the immediate release of the drug and a taste-masking agent comprising of the methacrylic acid copolymer. The taste-masked composition was obtained by spray drying of paroxetine and the polymer.

Patent Application WO 02/64119 discloses quickly disintegrating tablets in the oral cavity providing masking of the unpleasant taste and the fast absorption of the active from the tablets in the digestive tract. The disclosure is limited to the drug, which is hardly soluble in water under neutral or alkaline conditions but highly soluble in water under acidic conditions giving an unpleasant taste. The physicochemical properties of different drug molecules are different and so such systems would not be suitable for the drugs, which are water soluble. Patent Application WO 01/52848 discloses a taste masked oral formulation of linezolid which can be formulated as a suspension, a fast-disintegrating, effervescent or chewable tablet, by microencapsulating the antibiotic by solvent coacervation of ethyl cellulose with an optional seal coat of shellac and further coating the particles by functional polymer Eudragit L30 D. The formulated microcapsules can be suspended in an aqueous medium prior to oral administration to pediatric and geriatric patients, who are unwilling and/or find it difficult to swallow the tablets, else, fast-disintegrating tablets can be formulated which rapidly disperse into taste masked granules in the mouth.

Various methods for taste masking have been tried earlier, which include use of ion exchange resins, complexation of bitter drugs with pharmaceutically acceptable excipients and coating of drugs by lipids and various polymeric materials. Of these, the coating is the most widely used technique for taste masking. Coating of the active ingredient can be done by any of the techniques known in the art like microencapsulation, hot melt granulation, Fluid bed coating, and spray drying.

One of the approaches for taste masking is the use of ion exchange resins. Various anionic resins like Duolite AP143/1083 (cholestyramine resin USP), Cationic resin like Amberlite IRP 64 (copolymer of methacrylic acid crosslinked with divinylbenzene) and Dowex (based on polystyrenesulfonic acid crossliked with divinylbenzene) are used. US patent 6514492, discloses the use of ion exchange resin AMBERLITE.RTM. IRP 69 for taste masking of quinolone derivatives thereby eliminating the extreme bitterness of the quinolones in oral liquid formulation.

Patent Application WO 01/70194 discloses a fast dissolving orally consumable film adapted to adhere to and dissolve in the mouth of the consumer. The film is composed of an ion exchange resin, amberlite and a water soluble polymer pullulan as taste masking agent for the bitter drug, dextromethorphan. The film adheres to the oral cavity and dissolves to deliver the active ingredient. The use of the water soluble polymer in the formulation would restrict the use of such delivery system if the taste masking was desired for liquid oral preparation. Further such delivery systems may not be well accepted in case of pediatric and geriatric preparations where patient compliance is very important. U.S. Pat. No. 6,001,392 discloses a controlled release syrup suspension for the oral administration containing dextromethorphan adsorbed on to a polystyrene sulfonate ion exchange resin. The drug polymer complex is coated by a mixture of ethyl cellulose or ethyl cellulose latexes with plasticizers and water dispersible polymers such as SURELEASE. For the drugs where immediate release is required for rapid action, the controlled release of the active ingredient may not be favored and a delay in release may also be of concern for drugs having a limited absorption window.

The use of ion exchange resin to adsorb drugs containing amino groups for taste masking has found limited applicability in masking the taste of highly bitter drugs and also where the drug is to be dispersed in a liquid oral composition for long duration of time.

Complexation is yet another method for taste masking of bitter drugs. U.S. Pat. No. 4,808,411 discloses a taste masked composition comprising 75-95% of erythromycin and about 5 to 75% of carbomer where the drug and carbomer are held together by ionic interactions between erythromycin and carbomer. The complex is further coated with a functional polymer, hydroxy propyl methylcellulose phthalate to make the preparation palatable. Erythromycin is released slowly from the complex to avoid a significant perception of bitterness in the mouth. It is clear that slow release, not fast release of bitter medicament is critical as disclosed in the patent. But complexing alone is not sufficient enough to mask taste. Coating with functional polymers is required to attain desired palatability and further proper selection of complexing agent is vital since drug release should not be compromised.

Coating of drugs is another method but this alone may prove effective, only for moderately bitter drugs or in products where coated particles are formulated as aqueous preparations just before administration or are formulated in non-aqueous medium.

Patent Application WO 02/092106 discloses a taste-masked composition comprising polycarbophil and a macrolide antibiotic, clarithromycin. The complex is further coated with an acid resistant polymer Eudragit L100 55, releasing the drug in the intestine. For certain drugs the bioavailability may not be altered by the use of enteric coating where the drug is released in the small intestine, but for the drugs with a narrow absorption window restricted to the upper gastric region, the use of enteric coating may alter the bioavailability. European Patent Application EP 0409254 discloses an oral particulate preparation with unpleasant taste being masked using ethyl cellulose and a water swelling agent where the active is released rapidly from the said formulation. U.S. Pat. No. 5,635,200 discloses a taste-masked preparation of bitter drug ranitidine by a lipid coating and dispersion of these coated particles in the non-aqueous medium. U.S. Patent Application 2003-028025 discloses taste-masked composition of gatifloxacin suitable for use in oral dosage forms, particularly for pediatric formulations. A crystalline co-precipitate of gatifloxacin and one or both of stearic acid and palmitic acid is used to effectively mask the bitter taste of gatifloxacin in the mouth and in aqueous suspension through a full dosage cycle of fourteen days.

Patent Application WO 02/72111 discloses a taste masked pharmaceutical suspension of telithromycin. Four different coating agents Novata AB, Eudragit E100, glycerol monostearate and talc M10 are employed and at least three successive layers of coating are essential to taste mask telithromycin. The coated granules as disclosed could further be formulated as dry syrup, which is reconstituted as a suspension. U.S. Pat. No. 4,865,851 discloses yet another method for taste masking highly bitter 1 acetoxy ethyl ester of cefuroxime in particulate form being coated with an integral coating of lipid or a mixture of lipids, which serves to mask the taste.

The taste masking coatings, using lipids requires that the melting point of the lipid should be sufficiently high to prevent melting in the mouth and should not be so high that active ingredient itself melts or is chemically degraded. Cefuroxime axetil in a substantially amorphous form with maximum bioavailability has a low melting point of about 70 degree C. and the difference in the melting of the lipid and drug is very marginal and also the temperature at which the mixture is atomized is higher than the melting point of the lipid. The lipid based microencapsulation requires a highly sophisticated hot melt granulation process for producing fine particles without adversely affecting the drug molecule.

British Patent 2081092 also discloses a lipid coating for the purpose of taste masking. It was however found that wax coating resulted in poor dissolution of the active ingredients in the alimentary tract. Further the patent discloses a technique to overcome this problem by mixing the waxes with a water swellable polymer. Again the use of the water swellable polymer referred to in the patent makes it less appropriate for the liquid orals like suspensions and dry syrup. U.S. Pat. No. 5,286,489 describes a porous drug polymer matrix formed by admixing a bitter tasting active ingredient and a methacrylic ester copolymer in at least a 1:1 weight ratio of active ingredient to copolymer, effective to mask the taste of the drug. None of the examples described in the patent disclose the effect of these polymers on the release of the drug from the matrix. It is observed that the drug release is retarded from the matrix described herein.

Patent Application WO 00/56266 discloses the use of a high viscosity swellable polymer carbomer, in combination with film forming polymethacrylates and channelising agents for taste masking of bitter drugs. The addition of the water swellable polymer aids in the fast release of the active ingredient in the gastric media. In yet another Patent application WO 00/76479 a taste masking composition, using a combination of two enteric polymers comprising methacrylic acid copolymer and a phthalate polymer is disclosed. The patent discloses the use of the channelising agents which comprise the water soluble or water swellable materials to aid the release of the active ingredient The enteric polymers as disclosed in the patent are known to release the active ingredient in the alkaline pH where the polymers are soluble. Release of active ingredient will be delayed due to the use of the enteric polymers and in case of the medicaments having a narrow absorption window restricted to upper gastrointestinal tract; such system would be of limited use.

Microencapsulation of highly bitter drug cefuroxime axetil for taste masking is disclosed by M. Cuna et. al (M. Cuna, M. L. Lorenzo, J. L. Vila Jato, D. Torres, M. J. Alonso, Acta Technologiae et Legis Medicamenti. volume VII, N.3, 1996) using different polymeric materials like cellulose acetate trimellitate, HPMCP-50, HPMCP-55 with the final aim to mask the taste and assuring its release in the intestinal cavity.

In yet another publication by Alonso (M. J. Alonso, M. L Lorenzo-Lamosa, M. Cuna, J. L. Vila-Jato and D. Torres, Journal of Microencapsulation, 1997, Volume 14, No.5, 607-616) the encapsulation of cefuroxime axetil, a highly bitter drug, in pH sensitive acrylic microspheres in order to formulate a suspension dosage from is described. The acrylic polymers used were eudragit E, eudragit RL 100, eudragit L100-55. The cationic Polymer eudragit E showed a negative interaction with cefuroxime axetil. The enteric polymer eudragit L100-55 showed a favorable release in alkaline pH.

In the above disclosures the release of cefuroxime axetil was studied in the basic media whereas Dantzig et al (Anne H. Dantzig, Dale C. Duckworth, Linda B. Tabas, Biochimica et Biophysica Acta 1191, 1994, 7-13) showed that cefuroxime axetil is hydrolyzed to cefuroxime in the intestinal lumen by the esterases reducing the cefuroxime axetil concentration in the lumen and resulting in reduced absorption, leading to low bioavailability of Cefuroxime axetil in humans. Cefuroxime axetil already has a low bioavailability of 32-50% and hence further reduction in the bioavailability due to the formulation aspects should be minimized.

The taste masking formulations should be so designed that the bioavailability of the drugs is not compromised and the use of certain polymers like the enteric coatings should not affect the time to peak. Further the drug should be sufficiently absorbed to ensure effective therapeutic concentration in the plasma. Vogelman et al (B. Vogelman, William A. Craig Journal of Pediatric 1986, 108 (5, pt2) 835-40, & B. Vogelman, William A. Craig, S. Ebert, S. Gudmundsson, J. Leggett, Journal of Infectious Diseases 1988, 158(4), 831-47) have established that bactericidal killing is rapid, intensive and increases proportionately to the concentration. In the presence of high concentration of the drug, the killing is complete and almost instantaneous. In some drugs rapid and complete absorption and high systemic concentration are important to elicit the desired therapeutic effect.

There are certain drugs which pose challenges during the formulation due to their physico-chemical characteristics like cefuroxime axetil, a second generation cephalosporin antibiotic and celecoxib, from the class of COX 2 inhibitors. Both celecoxib and cefuroxime have relatively high dose requirement further increasing the difficulty in administering the therapeutically effective dose. Cefuroxime axetil exhibits the tendency to gel in contact with the aqueous media, necessitating that the dosage form disintegrates into particles rapidly and releases the drug at a faster rate before the gelling occurs in vivo. Another problem associated with cefuroxime relates to extremely bitter taste of the drug making it necessary to formulate cefuroxime in a coated delivery system to make it palatable. Celecoxib has an extremely low aqueous solubility and is not readily dissolved and dispersed for rapid absorption in the gastrointestinal tract further the amorphous form of celecoxib which is known to increase its dissolution and also its bioavailability tends to crystallize in contact with the aqueous medium. Etoricoxib, another molecule from the COX 2 inhibitor family is also associated with extremely bitter taste. Such active molecules which pose formulation problems and are required to be administered as rapid release formulations to overcome the low bioavailability, need to have a protective polymer coating which releases the active ingredient at a rapid rate without compromising the bioavailability, and masking the unpleasant taste of the active ingredient.

Patent Application WO 02/43707 discloses oral pharmaceutical formulations for cefuroxime axetil in tablet form such that the cefuroxime axetil is contained in the tablet core, coated with double layered film coat of hydroxypropyl Methyl cellulose and shellac. The first film coat as disclosed, serves to mask bitter taste of cefuroxime axetil and second film coat serves to delay the rupture time beyond 40 seconds. Since cefuroxime axetil is associated with gelling tendency in contact with aqueous media thereby reducing bioavailability, the rapid release of cefuroxime from the core of the dosage form is more desirable.

U.S. Pat. No. 5,599,556, discloses liquid formulations where the active ingredient is coated with single outer polymeric coating derived from prolamine cereal grain proteins and plasticizing agent. The bitter drug clarithromycin comixed with polyvinyl pyrrolidone is coated by prolamine to achieve taste masking and the coated particulate matter is dispersed in a suspending medium of pH greater than 6. The coatings are designed to rapidly degrade once the composition leaves the mouth and reaches the stomach. Most of the pharmaceutical liquid oral compositions are formulated at a pH of 3.5-5.5 (US Pharmacopoeia/National Formulary 23/NF 18, 1995). Some drugs may not be stable at the higher pH and some drugs may not be stable in extreme acidic pH and would tend to degrade over prolonged exposure.

U.S. Pat. No. 548,436 discloses chewable tablets made from a coated medicament where the coating is designed to be soluble at the lower pH of the stomach but relatively water insoluble at the higher pH of the mouth. The coatings comprise a polymer blend of dimethylaminoethyl methacrylate and neutral methacrylic acid ester and a cellulose ester. The above mentioned “reverse enteric” coating method of taste masking oral formulation is disclosed in connection with chewable tablets.

Patent Application WO 02/096392 discloses taste masking of highly water soluble drug cetrizine hydrochloride. The polymers like hydroxy propyl methyl cellulose, polyvinyl pyrrolidone, ethyl cellulose are used which effectively mask the taste of cetrizine in tablet form and release the drug immediately under the acidic conditions prevalent in stomach.

It is evident from the above disclosures, that taste masking can be achieved by various methods. Many natural and synthetic polymers, resins and waxes alone or in combination have been employed for taste masking. The enteric polymers like eudragit L are used for taste masking but the pH of saliva is near 5.8 and these polymers solubilize at pH beyond 5.5 so there is a possibility of drug being partially leached. It is understood that there is a need for the development of a taste masking polymer such that the bitter taste is completely masked by the polymer at the pH of saliva in mouth and in the reconstitution medium as in case of the liquid orals and further which is able to protect the drug in a biologically active form, from the moisture in the dosage form and releasing the drug rapidly in the stomach without affecting its absorption and bioavailability.

Whilst the use of polymer coats as mentioned in the above examples may be effective for retarding dissolution of the drug during the time in contact with saliva, during the process of swallowing, it has disadvantages in preparing taste masked liquid formulations intended for long term storage in contact with liquid medium. None of the references described above satisfactorily mask the bitter taste of the medicament in the pharmaceutical compositions like suspension, dry syrups where in the drug should not be leached in the suspension media up to 14 days, the normal reconstitution period and yet should be released in the gastric cavity immediately after ingestion without affecting the bioavailability. Regardless of the numerous techniques and pharmaceutical adjuncts known in the art to mask the taste of bitter-tasting medicaments, there remains the need to find an effective technique, adjunct or combination thereof for specific agents.

### OBJECTS OF THE INVENTION

It is the object of the present invention to provide an oral taste masked composition which can deliver a substantial amount of the bitter active immediately with improved palatability by using the specially synthesized pH sensitive polymers which solubilize or swell in the acidic conditions of the stomach and are insoluble or de-swell in the neutral or near neutral media and which can be applied in various pharmaceutical oral dosage forms. The term oral dosage form as used herein means any pharmaceutical composition intended to be administered to an individual by delivering said composition to the gastro intestinal tract of an individual via mouth. Oral dosage forms include tablets like chewable tablets, dispersible tablets, coated tablets; liquids such as dry syrups and suspensions.

The object of the present invention is to provide taste-masking compositions, consisting of a pH sensitive polymer and further a method for the synthesis of these polymers and also the method of preparation of pharmaceutical composition containing these polymers.

The other object of the present invention is to synthesize polymers, which effectively mask the unpleasant taste of the drug but do not compromise the dissolution rate and bioavailability of drug and further rapidly release the drug in the gastric cavity.

Yet another object of the present invention is to develop a pH sensitive polymer suitable for taste masking the liquid orals like suspensions, dry syrups, and solid dosage form like chewable tablets, fast dispersible tablets and conventional tablets.

Yet another object of the present invention is to prevent the leaching of the drug at the pH of saliva and in the reconstitution medium, from the liquid and solid dosage forms.

Yet another object of the present invention also aims at the coating of the bitter drug particle by various methods known in the art like microencapsulation, tray drying, fluid bed processing and spray drying etc.

A further object of the present invention is to formulate the liquid oral dosage forms comprising the coated particles.

### SUMMARY OF THE INVENTION

Accordingly the present invention provides for a taste masked pharmaceutical composition comprising a pH sensitive polymer and a bitter drug, and of the formula P[A₍ₓ₎B_{(y)}C_{(z)}]:D wherein P is the pH sensitive polymer comprising (A) a hydrophobic monomer, (B) a basic monomer and (C) a hydrophilic monomer and (D) a bitter drug and (x)=30-95%, (y)=5-70%, (z)=0-60%, all expressed in terms of w/w and the ratio of (P) to (D) is in the range of 30:1 to 0.2:1 w/w.

In one embodiment of the invention the hydrophobic monomer (A) is a acrylic or a methacrylic acid ester selected from the group consisting of cyclohexyl acrylate, dodecyl acrylate, 2 ethyl hexyl acrylate, octyl acrylate, tertiary butyl acrylate, phenyl acrylate, butyl acrylate, methyl methacrylate, benzyl methacrylate, cyclohexyl methacrylate, phenyl methacrylate, tertiary butyl methacrylate, butyl methacrylate, 2 ethyl hexyl methacrylate, propyl methacrylate preferably butyl acrylate, methyl methacrylate and butyl methacrylate.

In another embodiment of the invention the basic monomer (B) is selected from the group consisting of amino alkyl acrylic acid and methacrylic acid esters selected from the group consisting of dimethyl amino ethyl methacrylate, dimethyl amino ethyl acrylate, diethyl amino ethyl methacrylate, diethyl amino ethyl acrylate, piperidine ethyl methacrylate, 2 tert-butyl amino ethyl methacrylate, preferably dimethyl amino ethyl methacrylate and diethyl amino ethyl acrylate.

In still another embodiment of the invention the basic monomer (B) is an alkenyl pyridine selected from the group consisting of 2-vinyl pyridine, 3-vinyl pyridine, 4-vinyl pyridine and 5-vinyl 2 picoline, 2-vinyl 4 picoline, 2 isopropenyl pyridine, iso propenyl pyridine, preferably 4-vinyl pyridine.

In still another embodiment of the invention the basic monomer (B) is selected from vinyl quinolines, aminoalkyl vinyl ethers, amino ethyl styrenes and allylic amines, preferably allylic amines.

In yet another embodiment of the invention the hydrophilic monomer (C) is an acrylic or methacrylic acid ester selected from the group consisting of hydroxy ethyl methacrylate, hydroxy propyl methacrylate, hydroxy ethyl ethyl methacrylate, hydroxy ethyl acrylate, hydroxy propyl acrylate, hydroxy ethyl ethyl acrylate preferably hydroxy ethyl methacrylate and hydroxy ethyl ethyl methacrylate.

In one embodiment of the invention the drug comprises a macrolide antibiotic selected from the group consisting of erythromycin, azithromycin and clarithromycin, fluroquinolones selected from the group consisting of ciprofloxacin, enrofloxacin, ofloxacin, gatifloxacin, levofloxacin and norfloxacin, cephalosporins selected from the group consisting of cefuroxime, cephalexin, cephadroxil, cepfodoxime proxetil, nonsteoroidal, and anti-inflammatory and analgesic drugs selected from the group consisting of ibuprofen and diclofenac sodium and COX 2 inhibitors selected from the group consisting of etoricoxib and celecoxib, antihistamic drugs selected from the group consisting of chlorpheniramine maleate, oxazolidinones selected from the group consisting of linezolid and other drug like dextromethorphan.

In another embodiment of the invention, the total polymer to drug ratio for optimal taste masking bitter drug in the range 30:1 to 0.2:1 by weight. More preferably the ratio of the polymer to drug is 5:1 to 0.4:1 by weight.

In still another embodiment of the invention the pH sensitive polymer solubilizes or swells in the acidic pH ≦3 as found in stomach and remains insoluble or de swelled in the pH >3.5.

In yet another embodiment of the invention the drug itself or its pharmaceutically acceptable salt or ester or amide is used.

In another embodiment of the invention the drug is in the form of microparticles dispersed within or coated with the polymer matrix.

In yet another embodiment of the invention the pharmaceutical dosage forms which could be prepared using the composition of the present invention may be liquid orals; such as dry syrup or suspension and chewable or dispersible tablets.

In still another embodiment of the invention the pharmaceutical composition comprising the microparticles by themselves or in a pharmaceutically acceptable dosage form, release a minimal amount of drug at pH of saliva from the oral dosage form but rapidly release substantial amount of the drug immediately at pH ≦3 found in the stomach.

In the preferred embodiment the microparticles are formulated as aqueous suspension or are reconstituted in liquid medium for a normal storage period.

In yet another embodiment the pharmaceutical composition is obtained by dispersion or coating of the bitter drug in the matrix of pH sensitive polymer by any of the known techniques, preferably by microencapsulation, spray drying, fluid bed processing, co precipitation in a non solvent or by tray drying method.

In yet another embodiment the taste masked drug polymer matrix in particulate form is suspended using the reconstitution medium of pH 4.5 comprising of sucrose, tutti-frutti flavor, citric acid and polyvinyl pyrrolidone.

The present invention also relates to a process for the preparation of a a taste masked pharmaceutical composition comprising a pH sensitive polymer and a bitter drug, and of the formula P[A₍ₓ₎B_{(y)}C_{(z)}]:D wherein P is the pH sensitive polymer comprising (A) a hydrophobic monomer, (B) a basic monomer and (C) a hydrophilic monomer and (D) a bitter drug and (x)=30-95%, (y)=5-70%, (z)=0-60%, all expressed in terms of w/w and the ratio of (P) to (D) is in the range of 30:1 to 0.2:1 w/w.

In one embodiment of the invention the hydrophobic monomer (A) is a acrylic or a methacrylic acid ester selected from the group consisting of cyclohexyl acrylate, dodecyl acrylate, 2 ethyl hexyl acrylate, octyl acrylate, tertiary butyl acrylate, phenyl acrylate, butyl acrylate, methyl methacrylate, benzyl methacrylate, cyclohexyl methacrylate, phenyl methacrylate, tertiary butyl methacrylate, butyl methacrylate, 2 ethyl hexyl methacrylate, propyl methacrylate preferably butyl acrylate, methyl methacrylate and butyl methacrylate.

In another embodiment of the invention the basic monomer (B) is selected from the group consisting of amino alkyl acrylic acid and methacrylic acid esters selected from the group consisting of dimethyl amino ethyl methacrylate, dimethyl amino ethyl acrylate, diethyl amino ethyl methacrylate, diethyl amino ethyl acrylate, piperidine ethyl methacrylate, 2 tert-butyl amino ethyl methacrylate, preferably dimethyl amino ethyl methacrylate and diethyl amino ethyl acrylate.

In still another embodiment of the invention the basic monomer (B) is an alkenyl pyridine selected from the group consisting of 2-vinyl pyridine, 3-vinyl pyridine, 4-vinyl pyridine and 5-vinyl 2 picoline, 2-vinyl 4 picoline, 2 isopropenyl pyridine, iso propenyl pyridine, preferably 4-vinyl pyridine.

In still another embodiment of the invention the basic monomer (B) is selected from vinyl quinolines, aminoalkyl vinyl ethers, amino ethyl styrenes and allylic amines, preferably allylic amines.

In yet another embodiment of the invention the hydrophilic monomer (C) is an acrylic or methacrylic acid ester selected from the group consisting of hydroxy ethyl methacrylate, hydroxy propyl methacrylate, hydroxy ethyl ethyl methacrylate, hydroxy ethyl acrylate, hydroxy propyl acrylate, hydroxy ethyl ethyl acrylate preferably hydroxy ethyl methacrylate and hydroxy ethyl ethyl methacrylate.

In one embodiment of the invention the drug comprises a macrolide antibiotic selected from the group consisting of erythromycin, azithromycin and clarithromycin, fluroquinolones selected from the group consisting of ciprofloxacin, enrofloxacin, ofloxacin, gatifloxacin, levofloxacin and norfloxacin, cephalosporins selected from the group consisting of cefuroxime, cephalexin, cephadroxil, cepfodoxime proxetil, nonsteoroidal, and anti-inflammatory and analgesic drugs selected from the group consisting of ibuprofen and diclofenac sodium and COX 2 inhibitors selected from the group consisting of etoricoxib and celecoxib, antihistamic drugs selected from the group consisting of chlorpheniramine maleate, oxazolidinones selected from the group consisting of linezolid and other drug like dextromethorphan.

In yet another embodiment of the invention the drug itself or its pharmaceutically acceptable salt or ester or amide is used.

In another embodiment of the invention, the total polymer to drug ratio for optimal taste masking bitter drug in the range 30:1 to 0.2:1 by weight. More preferably the ratio of the polymer to drug is 5:1 to 0.4:1 by weight.

In still another embodiment of the invention the pH sensitive polymer solubilizes or swells in the acidic pH ≦3 as found in stomach and remains insoluble or de swelled in the pH >3.5.

In another embodiment of the invention the drug is in the form of microparticles dispersed within or coated with the polymer matrix.

### DETAILED DESCRIPTION OF THE INVENTION

The present invention provides oral pharmaceutical compositions, which effectively mask the bitter, unpleasant and otherwise undesirable taste of the active ingredient. More specifically the invention relates to the synthesis of the pH sensitive polymers which can be used in various pharmaceutical compositions providing taste masking and substantial immediate release and absorption of the bitter active ingredient, which is generally desirable in case of solid and liquid dosage forms like tablets; chewable or dispersible and suspensions or dry syrups. It also relates to the process for preparing such a composition.

The composition of the present invention is in the form of a taste masked formulation providing a substantial immediate release of the bitter active compound due to the solubilization or swelling of the reverse enteric polymer in acidic pH of ≦3 and the prevention of release of the drug in the pH range of >3.5 as found in saliva and reconstitution media over the complete period of storage of up to 14 days. The pH sensitive polymer is synthesized comprising of essentially of a hydrophobic monomer, a basic monomer and optionally a hydrophilic monomer.

An important feature of the present invention is that it provides taste-masked microcapsules of bitter drugs, suitable for oral administration as a suspension, a fast-disintegrating, effervescent or chewable tablet, and more specifically relates to such oral dosage forms in which the bitter taste of drugs is masked by a functional membrane coating on said microcapsules by pH sensitive polymer. A taste-masked microcapsule composition for taste masking an orally effective bitter drug in accordance with the present invention comprises microcapsules of the drug in a polymeric coating matrix prepared by emulsification, solvent evaporation or solvent extraction or by the spray drying technique. More specifically the present invention relates to the taste masked liquid oral formulation like the dry syrups intended for the pediatric use. It is likewise useful for preparations intended for all patients who, as a result of physical challenge or preference, would prefer a liquid preparation. The taste-masked compositions of the invention are further advantageous in that the reconstituted liquid preparations made from them are stable over the normal therapeutic dosage schedule, typically up to fourteen days.

According to the basic feature of the present invention, taste masking of bitter drug is achieved by using a pH sensitive polymeric coating on the bitter drug, wherein the polymer essentially solubilizes or swells in the acidic condition of the stomach and remains insoluble or de-swelled at neutral or near neutral pH. The pH sensitive polymer when applied to the pharmaceutical dosage forms like, the liquid orals such as dry syrup or suspension and tablets; chewable or dispersible, releases the active ingredient in the acidic pH of the stomach but maintains the taste palatable, by preventing the leaching of the drug in pH of saliva or suspending media or in the near neutral aqueous environment. The said pH sensitive polymers are synthesized using monomers essentially from the class of hydrophobic monomer and basic monomers and optionally a hydrophilic monomer.

Another aspect of the present invention is to formulate a coated bitter drug in the form of suspensions and prevent leaching of the drug in suspending media during reconstitution period of up to 14 days, and also ensure substantial release of active drug in the simulated gastric fluid without compromising on bioavailability. pH sensitive polymers of the present invention inhibit the release of the active agent in the aqueous media of pH >3.5 such that the leaching of bitter drug in the saliva and also in the reconstitution media, in case of liquid orals is inhibited and release the drug rapidly in the pH range of ≦3 as found in the stomach.

The present invention also provides for the taste masking of bitter drugs like macrolide antibiotics such as erythromycin, azithromycin and clarithromycin, fluroquinolones such as ciprofloxacin, enrofloxacin, ofloxacin, gatifloxacin, levofloxacin and norfloxacin, cephalosporins such as cefuroxime, cephalexin, cephadroxil, cepfodoxime proxetil, nonsteoroidal and anti-inflammatory and analgesic drugs such as ibuprofen, diclofenac sodium and COX 2 inhibitors like etoricoxib and celecoxib, antihistamic drugs like chlorpheniramine maleate, oxazolidinones like linezolid and other drug like dextromethorphan. The drug itself or its pharmaceutically acceptable salt or ester or amide may be used in the present invention. The drugs preferred for the practice of present invention can be chosen from a wide range comprising cefuroxime axetil, ciprofloxacin, celecoxib and clarithromycin. The pharmaceutical composition described herein has the total polymer to drug ratio for optimal taste masking bitter drug in the range 30:1 to 0.2:1 by weight. More preferably the ratio of the polymer to drug is 5:1 to 0.4:1 by weight.

In particular, the invention comprises development of a formulation useful as a stable taste-masking liquid suspension capable of being ingested without producing the unpleasant taste associated with the active agent, while still providing immediate bioavailability upon exposure to the pH levels found in the stomach of a human.

In another feature the taste-masked particles obtained as described in the invention are optionally blended with other pharmaceutically acceptable excipients such as flavors, sweeteners, suspending agents and/or preservatives and formulated as dry syrup or compressed into fast disintegrating, effervescent or chewable tablets. Stable aqueous suspensions can be constituted from the dry syrup powder for oral administration up to 14 days for pediatric and geriatric patients who are unwilling and/or find it difficult to swallow tablets. Fast disintegrating tablets rapidly disintegrate in the mouth and are therefore suitable for oral administration to patients who find it difficult to swallow tablets. Such dosage forms on oral administration should release not more than 10%, most preferably not more than 5%, at pH of reconstitution media up to 14 days but rapidly release at least 40-60% within 15 min and not less than 70% in an hour at pH as found in the stomach.

The mean particle size of the microcapsules will be in the range of about 30 to 1000 microns, most preferably in the range of about 100 to 500 microns.

Examples of the bitter, unpleasant tasting drugs which may be used include, but are not limited to macrolide antibiotics such as erythromycin azithromycin and clarithromycin, fluroquinolones such as ciprofloxacin enrofloxacin, ofloxacin, gatifloxacin, levofloxacin and norfiloxacin, cephalosporins such as cefuroxime, cephalexin, cephadroxil, cepfodoxime proxetil nonsteoroidal and anti-inflammatory and analgesic drugs such as ibuprofen, diclofenac sodium and COX 2 inhibitors like celecoxib and etoricoxib, antihistamic drugs like chlorpheniramine maleate, oxazolidinones like linezolid and other drug like dextromethorphan.

Various derivatives of polymethacrylates and cellulose polymers have been used in the past to coat antibiotic drug cefuroxime axetil. The release profile of the drug from these materials depends on the type of polymer being used for the coating. The patent WO0236126 discloses a sustained release of cefuroxime axetil from the composition comprising of eudragit L 30 D, eudragit RL 30 D and Eudragit RS 30D. The release of cefuroxime axetil in 0.07N HCl for 1 hr and phosphate buffer of pH 6.8 is disclosed as follows:

| Time: | 60 min | 120 min | 180 min | 240 min | 360 min |
|---|---|---|---|---|---|
| % release: | 34.6 | 44.3 | 67.4 | 83.7 | 96.1 |

Alonso disclosed the release of cefuroxime axetil from the microparticles obtained using the polymeric coating of Eudragit E, Eudragit L100-55 and eudragit RL-100. (M. J. Alonso, M. L Lorenzo-Lamosa, M. Cuna, J. L. Vila-Jato and D. Torres, Journal of Microencapsulation, 1997, Volume 14, No.5, 607-616). The release of cefuroxime axetil was almost complete from eudragit E microspheres in 0.07 N HCl in 20-30 min. The release from the eudragit E microsphere in Sorensens buffer pH 7 was found to be slow as compared to the 0.07 N HCl. The release data are summarized below.

| | Media 0.07 N HCL | | | | | |
|---|---|---|---|---|---|---|
| Time: | 10 min to 20 min | | | | | |
| % release: upto | 80 to 100 | | | | | |
| | Media pH 5.8 Sorensens buffer | | | | | |
| Time: | 10 min | 20 min | 30 min | 40 min | 50 min | 60 min |
| % release: upto | 20 | 30 | 40 | 45 | 50 | 60 |

The cationic Polymer eudragit E however showed a negative interaction with cefuroxime axetil showing a significant degradation in presence of Eudragit E. The microspheres using eudragit L 55 and RL showed a release of <9% in 2 hrs in pH 5.2 and a significant amount of 75% released in 30 min at pH 6.0.

M. Cuna et. al discloses the coating of cefuroxime axetil for taste masking (M. Cuna, M. L. Lorenzo, J. L. Vila Jato, D. Torres, M. J. Alonso, Acta Technologiae et Legis Medicamenti. volume VII, N.3, 1996) using different polymeric materials like cellulose acetate trimellitate, HPMCP-50, HPMCP-55. The release data:are summarized below

| | (microspheres with HPMCP-55) At pH 5.2 | |
|---|---|---|
| Time: | 15 min to 60 min | |
| % release: | less than 25% | |
| | (microspheres with HPMCP-55) At pH 6.0 | |
| Time: | 15 min | 30 min |
| % release: | 50-75 | 75-100 |

In the above disclosures the release of cefuroxime axetil was studied in the basic media whereas Dantzig et al (Anne H. Dantzig, Dale C. Duckworth, Linda B. Tabas, Biochimica et Biophysica Acta 1191, 1994, 7-13) showed that cefuroxime axetil is hydrolyzed to cefuroxime in the intestinal lumen by the esterases reducing the cefuroxime axetil concentration in the lumen and resulting in reduced absorption, leading to low bioavailability of Cefuroxime axetil in humans.

The drug molecules like cefuroxime axetil tend to gel in presence of the aqueous media. Also if the tablets are not protected from moisture during storage, they result in poor dissolution and lower drug bioavailability. So the liquid oral preparation of cefuroxime axetil needs to protect the drug during the reconstitution period from the aqueous environment. Cefuroxime axetil has a limited absorption region in the gastrointestinal tract as the enzyme esterases, hydrolyses it to cefuroxime, which cannot be absorbed across the tract thereby reducing its bioavailability. Cefuroxime axetil is also associated with an extremely bitter taste. The pharmaceutical compositions of cefuroxime axetil are therefore required to be taste masked. The use of water soluble and enteric coating polymers for Cefuroxime axetil are therefore of limited use. In the preferred embodiment of the present invention the bitter drugs used for taste masking include cefuroxime axetil, ciprofloxacin hydrochloride and clarithromycin. One of the feature of the present invention is fast swelling and/or dissolution of the polymer in acidic pH, with rapid release of drug, like cefuroxime axetil which have a low bioavailability of 32-50%, from the polymer coating and thus should not cause any delay in the absorption and alter the bioavailability. The release of the drugs form the polymers used in the present invention is disclosed in the examples.

In another feature of the present invention the pharmaceutical composition may be obtained by coating of the drug using of pH sensitive polymer either by microencapsulation, spray drying, fluid bed processing, co-precipitation in a non solvent or by tray drying method. The drug is dispersed within the polymer matrix.

In still another feature the taste masking compositions are made by microencapsulation of the drug in the polymer matrix. The microencapsulation of the bitter drugs can be obtained by emulsification, solvent evaporation or solvent extraction and spray drying of the drug polymer solution or dispersion of drug in polymer solution. If the drug is not soluble in the polymer solution then it is dispersed in the polymer solution uniformly with the help of the dispersing agents like the surfactants. The preferred surfactants are the nonionic surfactants belonging to the class of SPAN and TWEEN. Preferably the solvent is selected such that the drug and the polymer are both soluble in the solvent. In the preferred embodiment of the present invention the solvents chosen for the solubilization of the drug and polymer are alcohols like methanol, ethanol, isopropanol, butanol, chlorinated hydrocarbons like dichloromethane, chloroform, ketones like methyl ethyl ketone, methyl iso-butyl ketone and acetone. Preferably the solvents used to dissolve the drug and polymers are methanol, acetone and dichloromethane. The preferred solvent to dissolve the drug and polymer is acetone or a mixture of methanol and dichloromethane, in the ratio 1:1 to 1:1.5.

The taste-masked microcapsules of the bitter drug can be obtained by microencapsulation by emulsification solvent evaporation technique. The dispersed phase is the organic solvent containing the drug and polymer and the dispersion medium is the liquid paraffin. The pH sensitive polymer synthesized is dissolved in the organic solvent (acetone, methanol, dichloromethane or a mixture of methanol and dichloromethane in the ratio 1:1 to 1:1.5.) The drug is added to the polymer solution resulting in a solution or a homogeneous dispersion. The organic phase is then added into the light liquid paraffin-containing span 85 (0.1 to 1% w/w). A constant mechanical stirring rate of 1000 rpm and at room temperature is maintained for a 3-4 hours. The solvent is allowed to evaporate and the microspheres so obtained are separated by filtration, washed by petroleum ether or by n hexane and dried under vacuum for up to 24 hours.

The taste-masked microcapsules of the bitter drug can be further obtained by microencapsulation by emulsification solvent extraction technique. The dispersed phase is the organic solvent containing the drug and polymer and the dispersion medium is the liquid paraffin. The pH sensitive polymer synthesized is dissolved in the organic solvent (acetone, methanol, dichloromethane or a mixture of methanol and dichloromethane in the ratio 1:1 to 1:1.5.). The drug is added to the polymer solution resulting in a solution or a homogeneous dispersion. The organic phase is then added into the light liquid paraffin-containing span 85 (0.1 to 1% w/w). A constant mechanical stirring rate of 500 rpm and 25° C. is maintained for 30 min and 40 ml of n hexane or cyclohexane is added at a rate of 5 ml/min, followed by another 40 ml n hexane or cyclohexane being added rapidly. The agitation is maintained for another 10-15 min then the microparticles are separated by filtration and washed by petroleum ether or by n hexane and dried at 27° C. under vacuum for up to 24 hours.

Alternately the taste masked micro particles can be obtained by spray drying. The drug-polymer solution or dispersion in the organic solvent is spray dried to obtain the taste masked micro particles. The drying gas can be an inert gas such as nitrogen, argon and carbon dioxide or air. The preferred gas in the present invention is air. The gas inlet temperature to the spray dryer depends on the choice of the solvent used but may be in the range of 35-150° C. preferably 40-60° C. The gas outlet temperature is similarly dependant on the solvent but may be in the range of 25 to 50, preferably 25 to 40° C. The polymer is solubilized in methanol or a mixture of methanol and dichloromethane 1:1 and the drug is either solubilized or dispersed in the polymer solution. The resulting mixture is spray dried to obtain the taste masked micro particles.

The taste masked particles and granules obtained may be mixed with the flavoring agents such as natural or artificial flavors, citric and tartaric acids, sweeteners such as sucrose, saccharin and aspartame, and other pharmaceutically acceptable excipients to be formulated as conventional whole, chewable or dispersible tablets, dry syrups, suspensions, sachets or any other suitable oral dosage form.

The present invention is more directed towards the taste masking of the liquid oral compositions suitable for the pediatric patients or those, who have a difficulty in swallowing the solid dosage form. The taste masked pharmaceutical composition is prepared by reconstitution of the polymer coated drug particles in a liquid vehicle comprising sucrose, flavor and citric acid and a suspending agent like cellulose derivatives or polyvinyl pyrrolidone or xanthan gum etc. The taste masked pharmaceutical composition of the present invention is prepared by using the reconstitution medium of pH 4.5 comprising of sucrose, tutti-frutti flavor, citric acid and polyvinyl pyrrolidone.

The taste masked pharmaceutical compositions as exemplified in the examples 1 to 12 given below were tested for the drug release with respect to time. Cefuroxime axetil release from the taste masked particles was determined in 900 ml of 0.07 N hydrochloric acid, at 37±0.5° C., using USP type II apparatus rotated at 100 rpm. The samples were withdrawn at 15, 30, 45, 60 and 90 min. The amount withdrawn each time was replaced with fresh media to maintain the sink conditions.

Ciprofloxacin hydrochloride release from the taste masked particles was determined in 900 ml of 0.1 N hydrochloric acid buffer, at 37±0.5° C., using USP type II apparatus rotated at 100 rpm. The samples were withdrawn at 15, 30, 45, and 60, min. The amount withdrawn each time was replaced with fresh media to maintain the sink conditions.

Clarithromycin release from the taste masked particles was determined in 900 ml of acetate buffer pH 2.8, at 37±0.5° C., using USP type II apparatus rotated at 100 rpm. The samples were withdrawn at 15, 30, 45 and 60 min. The amount withdrawn each time was replaced with fresh media to maintain the sink conditions.

Celecoxib release from taste masked particles was determined by placing composition consisting of celecoxib and polymer in 0.1 N HCl 100 ml for 30 min and then addition of 900 ml of 0.1 N NaOH solution at 37±0.5° C., using USP type II apparatus rotated at 100 rpm. Samples were withdrawn from 0.1 N NaOH solution at 15, 30, 45 and 60 min. Amount withdrawn each time was replaced with fresh media to maintain sink conditions.

Taste making compositions and properties thereof are described below with reference to illustrative examples which should not be construed to limit the scope of the present invention in any manner.

### EXAMPLE 1

Taste masked microcapsules were obtained by emulsification solvent evaporation technique. 3.50 g of ciprofloxacin was dispersed in polymer solution containing 900 mg of polymer in 45 ml of mixture of methanol and dichloromethane (1:1). The polymer has the monomer composition Methyl methacrylate 60% by weight Hydroxyethyl methacrylate 25% by weight and Vinyl Pyridine 15% by weight. The nonionic surfactant Span 85 was added 0.5% w/w to facilitate the dispersion of ciprofloxacin in the polymer solution. The dispersion of ciprofloxacin was added dropwise to the bath of light liquid paraffin under mechanical stirring. A constant mechanical stirring rate of 1000 rpm and at room temperature was maintained for a 3-4 hours. Solvent was allowed to evaporate and the microspheres so obtained were separated by filtration, washed by petroleum ether and dried at 27° C. under vacuum for 24 hours. Drug release pattern of the composition prepared was studied and results are tabulated in Table-1. Results in Table-1 show the immediate release of the drug.

TABLE 1

| Time (min) | % Release |
|---|---|
| 15 | 86.58 |
| 30 | 91.57 |
| 45 | 96.85 |

### EXAMPLE 2

Taste-masked microcapsules of the bitter drug were obtained by microencapsulation by emulsification solvent evaporation technique. 2.35 g of ciprofloxacin was dispersed in polymer solution containing 7.0 g of polymer in 40 ml of mixture of methanol and dichloromethane (1:1). The polymer has the monomer composition Methyl methacrylate 60% by weight Hydroxyethyl methacrylate 25% by weight and Vinyl Pyridine 15% by weight. The microencapsulation of the ciprofloxacin with the pH sensitive polymer was achieved using the method similar as mentioned in the example 1. The drug release pattern of the composition prepared was studied and the results are tabulated in Table-2

TABLE 2

| Time (min) | % Release |
|---|---|
| 15 | 61.45 |
| 30 | 68.30 |
| 45 | 74.56 |
| 60 | 81.42 |

### EXAMPLE 3

Taste masked microcapsules were obtained by microencapsulation by emulsification solvent evaporation technique. 2.0 g of clarithromycin was dissolved in polymer solution containing 4.0 g of polymer in 40 ml of mixture of methanol and dichloromethane (1:1). The polymer has the monomer composition Methyl methacrylate 60% by wt Hydroxyethyl methacrylate 25% by wt and Vinyl Pyridine 15% by wt. Microencapsulation of clarithromycin with pH sensitive polymer was achieved using a method similar to that of example 1. Drug release pattern of composition prepared was studied and results are tabulated in Table 3.

TABLE 3

| Time (min) | % Release |
|---|---|
| 15 | 48.20 |
| 30 | 65.51. |
| 45 | 72.80 |
| 60 | 82.59 |

### EXAMPLE 4

Taste masked microcapsules were obtained by microencapsulation by emulsification solvent evaporation technique. 2.0 g of clarithromycin was dissolved in polymer solution containing 1.2 g of polymer in 30 ml of mixture of methanol and dichloromethane (1:1). The polymer has the monomer composition Methyl methacrylate 60% by wt, Hydroxyethyl methacrylate 25% by wt and Vinyl Pyridine 15% by wt. Microencapsulation of clarithromycin with pH sensitive polymer was achieved using a method similar to that of example 1. Drug release pattern of composition prepared was studied and results are tabulated in Table 4

TABLE 4

| Time (min) | % Release |
|---|---|
| 15 | 53.97 |
| 30 | 69.40 |
| 45 | 76.32 |
| 60 | 85.59 |

### EXAMPLE 5

Taste masked microcapsules of cephalosporin antibiotic cefuroxime axetil were obtained by microencapsulation by emulsification solvent evaporation technique. 2.0 g of cefuroxime axetil was dissolved in polymer solution containing 6.0 g of polymer in 40 ml of mixture of methanol and dichloromethane (1:1). The polymer has the monomer composition Methyl methacrylate 43% by wt Hydroxyethyl methacrylate 42% by wt and Vinyl Pyridine 15% by wt. Microencapsulation of cefuroxime axetil with pH sensitive polymer was achieved using a method similar to that of example 1. Drug release pattern of composition prepared was studied and results are tabulated in Table-5

TABLE 5

| Time (min) | % Release |
|---|---|
| 15 | 95.2 |
| 30 | 97.2 |

Taste masked pharmaceutical composition of microcapsules prepared in example S is prepared for microparticles having drug equivalent to 4 doses by using reconstitution medium of pH 4.5 comprising of sucrose 85% w/v, tutti-frutti flavor qs., citric acid qs. and polyvinyl pyrrolidone 2%. The drug release during the storage for 7 days is shown in the table 6

TABLE 6

| Day | % Release |
|---|---|
| 2 | 0.26 |
| 3 | 0.31 |
| 4 | 0.38 |
| 5 | 0.50 |
| 6 | 0.60 |
| 7 | 0.68 |

### EXAMPLE 6

Taste masked microcapsules of cefuroxime axetil were obtained by microencapsulation by emulsification solvent evaporation technique. 2.0 g of cefuroxime axetil was dissolved in polymer solution containing 6.0 g of polymer in 40 ml of mixture of methanol and dichloromethane 1:1). The polymer has the monomer composition Methyl methacrylate 60% by weight, Hydroxyethyl methacrylate 25% by weight and Vinyl Pyridine 15% by weight. The microencapsulation of cefuroxime axetil with the pH sensitive polymer was achieved using the method similar as mentioned in the example 1. The drug release pattern of the composition prepared was studied and the results are tabulated in Table-7

TABLE 7

| Time (min) | % Release |
|---|---|
| 15 | 78.72 |
| 30 | 80.52 |
| 45 | 88.2 |
| 60 | 95.12 |

Taste masked pharmaceutical composition of microcapsules prepared in example 6 is prepared for microparticles having drug equivalent to 4 doses by using reconstitution medium of pH 4.5 comprising of sucrose 85% w/v, tutti-frutti flavor qs., citric acid qs. and polyvinyl pyrrolidone 2%. The drug release during the storage for 7 days is shown in the table 8

TABLE 8

| Day | % Release |
|---|---|
| 2 | 0.25 |
| 3 | 0.27 |
| 4 | 0.38 |
| 5 | 0.45 |
| 6 | 0.57 |
| 7 | 0.64 |

### EXAMPLE 7

Taste masked microcapsules of the bitter cephalosporin antibiotic cefuroxime axetil were obtained by microencapsulation by emulsification solvent evaporation technique. 2.0 g of cefuroxime axetil was dissolved in polymer solution containing 6.0 g of polymer in 40 ml of mixture of methanol and dichloromethane (1:1). The polymer has the monomer composition Methyl methacrylate 70% by weight and Vinyl Pyridine 30% by weight. The microencapsulation of the cefuroxime axetil with the pH sensitive polymer was achieved using the method similar as mentioned in the example 1. The drug release pattern of the composition prepared was studied and the results are tabulated in Table-9

TABLE 9

| Time (min) | % Release |
|---|---|
| 15 | 92.7 |
| 30 | 96.2 |
| 45 | 97.3 |

Taste masked pharmaceutical composition of microcapsules prepared in example 7 is prepared for microparticles having drug equivalent to 4 doses by using reconstitution medium of pH 4.5 comprising of sucrose 85% w/v, tutti-frutti flavor qs., citric acid qs. and polyvinyl pyrrolidone 2%. The drug release during the storage for 7 days is shown in the table 10

TABLE 10

| Day | % Release |
|---|---|
| 2 | 0.76 |
| 3 | 1.32 |
| 4 | 1.71 |
| 5 | 1.73 |
| 6 | 2.10 |
| 7 | 2.14 |

### EXAMPLE 8

Taste masked microcapsules of the bitter cephalosporin antibiotic cefuroxime axetil were obtained by microencapsulation by emulsification solvent evaporation technique. 2.0 g of cefuroxime axetil was dissolved in polymer solution containing 6.0 g of polymer in 40 ml of mixture of methanol and dichloromethane (1:1). The polymer has the monomer composition Methyl methacrylate 35% by weight, Hydroxyethyl methacrylate 35% by weight and Vinyl Pyridine 30% by weight. The microencapsulation of the cefuroxime axetil with the pH sensitive polymer was achieved using the method similar as mentioned in the example 1. The drug release pattern of the composition prepared was studied and the results are tabulated in Table-11

TABLE 11

| Time (min) | % Release |
|---|---|
| 15 | 91.85 |
| 30 | 95.38 |
| 45 | 97.53 |

### EXAMPLE 9

The cefuroxime axetil-polymer solution in the organic solvent was spray dried to obtain the taste masked micro particles. The polymer has the monomer composition Methyl methacrylate 60% by weight Hydroxyethyl methacrylate 25% by weight and 4 Vinyl pyridine 15% by weight of polymer The drying gas was air. The inlet air temperature to the spray dryer was in the range 40-70° C. The outlet air temperature was in the range of 25 to 60° C. The polymer weighing 2.4 g was solubilized in the mixture of methanol and dichloromethane 1:1 and cefuroxime axetil weighing 4.8 g was added in the polymer solution. The atomization was in the range of 1-2 kg. The feed rate was 20 to 85 rpm The resulting solution was spray dried to obtain the taste masked micro particles. The drug release pattern of the composition prepared was studied and the results are tabulated in Table-12

TABLE 12

| Time (min) | % Release |
|---|---|
| 15 | 53.47 |
| 30 | 69.97 |
| 45 | 84.47 |
| 60 | 91.72 |
| 90 | 93.18 |

Taste masked pharmaceutical composition of microcapsules prepared in example 9 is prepared for microparticles having drug equivalent to 5 doses by using reconstitution medium of pH 4.5 comprising of sucrose 85% w/v, tutti-frutti flavor qs., citric acid qs. and polyvinyl pyrrolidone 2%. The drug release during the storage for 7 days is shown in the table 13

TABLE 13

| Day | % Release |
|---|---|
| 2 | 0.96 |
| 3 | 1.43 |
| 4 | 1.92 |
| 5 | 2.57 |
| 6 | 2.88 |
| 7 | 3.44 |

### EXAMPLE 10

The cefuroxime axetil-polymer solution in the organic solvent was spray dried to obtain the taste masked micro particles. The polymer has the monomer composition Methyl methacrylate 60% by weight Hydroxyethyl methacrylate 25% by weight and 4 Vinyl pyridine 15% by weight of polymer The solvent used was a mixture of methanol and dichloromethane 1:1. The drying gas was air. The inlet air temperature to the spray dryer was in the range 40-70° C. The outlet air temperature was in the range of 25 to 60° C. The polymer weighing 2.4 g was solubilized in the in the mixture of methanol and dichloromethane 1:1 and cefuroxime axetil weighing 4.8 g was added in the polymer solution. The atomization was in the range of 1-2 kg. The feed rate was 20 to 85 rpm. The resulting solution was spray dried to obtain the taste masked micro particles. The drug release pattern of the composition prepared was studied and the results are tabulated in Table-14

TABLE 14

| Time (min) | % release |
|---|---|
| 15 | 72.58 |
| 30 | 85.41 |
| 45 | 89.48 |
| 60 | 93.8 |
| 90 | 94.58 |

The taste masked pharmaceutical composition of the microcapsules prepared in example 10 is prepared for microparticles having the drug equivalent to 5 doses by using the reconstitution medium of pH 4.5 comprising of sucrose 85% w/v, tutti-frutti flavor qs., citric acid qs. and polyvinyl pyrrolidone 2%. The drug release during the storage for 7 days is shown in the table 15

TABLE 15

| Day | % Release |
|---|---|
| 2 | 0.85 |
| 3 | 1.36 |
| 4 | 2.26 |
| 5 | 3.0 |
| 6 | 3.44 |
| 7 | 3.84 |

### EXAMPLE 11

The celecoxib-polymer solution in the organic solvent was spray dried to obtain the taste masked micro particles. The polymer has the monomer composition Methyl methacrylate 60% by weight Hydroxyethyl methacrylate 25% by weight and 4 Vinyl pyridine 15% by weight of polymer. The solvent used was a mixture of methanol and dichloromethane (1.5:1). The drying gas was air. The inlet air temperature to the spray dryer was in the range 40-70° C. The outlet air temperature was in the range of 25 to 60° C. The polymer weighing 1.5 g was solubilized in mixture of methanol and dichloromethane (1.5:1) and celecoxib weighing 2.0 g was added in the polymer solution. The atomization was in the range of 1-2 kg. The feed rate was 20 to 85 rpm. The resulting solution was spray dried to obtain the taste masked micro particles. The drug release pattern of the composition prepared was studied and the results are tabulated in Table-16

TABLE 16

| Time (min) | % Release |
|---|---|
| 15 | 86.8 |
| 30 | 94.0 |

### EXAMPLE 12

The celecoxib-polymer solution in the organic solvent was spray dried to obtain the taste masked micro particles. The polymer has the monomer composition Methyl methacrylate 60% by weight Hydroxyethyl methacrylate 25% by weight and 4 Vinyl pyridine 15% by weight of polymer The solvent used was a mixture of methanol and dichloromethane (1.5:1). The drying gas was air. The inlet air temperature to the spray dryer was in the range 40-70° C. The outlet air temperature was in the range of 25 to 60° C. The polymer weighing 0.750 g was solubilized in the mixture of methanol and dichloromethane (1.5:1) and celecoxib weighing 2.0 g was added in the polymer solution. The atomization is in the range of 1-2 kg. The feed rate was 20 to 85 rpm The resulting solution was spray dried to obtain the taste masked micro particles. The drug release pattern of the composition prepared was studied and the results are tabulated in Table-17

TABLE 17

| Time (min) | % Release |
|---|---|
| 15 | 73.45 |
| 30 | 93.74 |

1. A taste masked pharmaceutical composition comprising a pH sensitive polymer and a bitter drug, and of the formula P[A₍ₓ₎B_{(y)}C_{(z)}]:D wherein P is the pH sensitive polymer comprising (A) a hydrophobic monomer, (B) a basic monomer and (C) a hydrophilic monomer and (D) a bitter drug and (x)=30-95%, (y)=5-70%, (z)=0-60%, all expressed in terms of w/w and the ratio of (P) to (D) is in the range of 30:1 to 0.2:1 W/W.

2. A composition as claimed in claim 1 wherein the hydrophobic monomer (A) is a acrylic or a methacrylic acid ester selected from the group consisting of cyclohexyl acrylate, dodecyl acrylate, 2 ethyl hexyl acrylate, octyl acrylate, tertiary butyl acrylate, phenyl acrylate, butyl acrylate, methyl methacrylate, benzyl methacrylate, cyclohexyl methacrylate, phenyl methacrylate, tertiary butyl methacrylate, butyl methacrylate, 2 ethyl hexyl methacrylate, propyl methacrylate preferably butyl acrylate, methyl methacrylate and butyl methacrylate.

3. A composition as claimed in claim 1 wherein the basic monomer (B) is selected from the group consisting of amino alkyl acrylic acid and methacrylic acid esters selected from the group consisting of dimethyl amino ethyl methacrylate, dimethyl amino ethyl acrylate, diethyl amino ethyl methacrylate, diethyl amino ethyl acrylate, piperidine ethyl methacrylate, 2 tert-butyl amino ethyl methacrylate.

4. A composition as claimed in claim 3 wherein the basic monomer (B) is selected from dimethyl amino ethyl methacrylate and diethyl amino ethyl acrylate

5. A composition as claimed in claim 1 wherein the basic monomer (B) is an alkenyl pyridine selected from the group consisting of 2-vinyl pyridine, 3-vinyl pyridine, 4-vinyl pyridine, 5-vinyl 2 picoline, 2-vinyl 4 picoline, 2 isopropenyl pyridine and iso propenyl pyridine.

6. A composition as claimed in claim 5 wherein the basic monomer (B) is 4-vinyl pyridine.

7. A composition as claimed in claim 1 wherein the basic monomer (B) is selected from vinyl quinolines, aminoalkyl vinyl ethers, amino ethyl styrenes and allylic amines.

8. A composition as claimed in claim 7 wherein the basic monomer (B) is an allylic amine.

9. A composition as claimed in claim 1 wherein the hydrophilic monomer (C) is an acrylic or methacrylic acid ester selected from the group consisting of hydroxy ethyl methacrylate, hydroxy propyl methacrylate, hydroxy ethyl ethyl methacrylate, hydroxy ethyl acrylate, hydroxy propyl acrylate, hydroxy ethyl ethyl acrylate.

10. A composition as claimed in claim 1 wherein the hydrophilic monomer (C) is selected from hydroxy ethyl methacrylate and hydroxy ethyl ethyl methacrylate.

11. A composition as claimed in claim 1 wherein the drug comprises a macrolide antibiotic selected from the group consisting of erythromycin, azithromycin and clarithromycin, fluroquinolones selected from the group consisting of ciprofloxacin, enrofloxacin, ofloxacin, gatifloxacin, levofloxacin and norfloxacin, cephalosporins selected from the group consisting of cefuroxime, cephalexin, cephadroxil, cepfodoxime proxetil, nonsteoroidal, and anti-inflammatory and analgesic drugs selected from the group consisting of ibuprofen and diclofenac sodium and COX 2 inhibitors selected from the group consisting of etoricoxib and celecoxib, antihistamic drugs selected from the group consisting of chlorpheniramine maleate, oxazolidinones selected from the group consisting of linezolid and other drug like dextromethorphan.

12. A composition as claimed in claim 1 wherein the drug itself or its pharmaceutically acceptable salt or ester or amide is used.

13. A composition as claimed in claim 1 wherein the total polymer to drug ratio is in the range 30:1 to 0.2:1 by weight.

14. A composition as claimed in claim 1 wherein the total polymer to drug ratio is in the range of 5:1 to 0.4:1 by weight.

15. A composition as claimed in claim 1 wherein the drug is in the form of microparticles dispersed within or coated with the polymer matrix.

16. A composition as claimed in claim 1 wherein the pH sensitive polymer solubilizes or swells in the acidic pH ≦3 as found in stomach and remains insoluble or deswelled in the pH >3.5.

17. A composition as claimed in claim 1 wherein the pharmaceutical dosage forms which could be prepared using the composition of the present invention is selected from liquid orals comprising dry syrup or suspension and chewable or dispersible tablets.

18. A composition as claimed in claim 1 wherein the pharmaceutical composition comprising the microparticles by themselves or in a pharmaceutically acceptable dosage form, release a minimal amount of drug at pH of saliva from the oral dosage form but rapidly release substantial amount of the drug immediately at pH ≦3 found in the stomach.

19. A composition as claimed in claim 1 wherein the microparticles are formulated as aqueous suspension or are reconstituted in liquid medium for a normal storage period.

20. A composition as claimed in claim 1 wherein the pharmaceutical composition is obtained by dispersion or coating of the bitter drug in the matrix of pH sensitive polymer by any of the known techniques, preferably by microencapsulation, spray drying, fluid bed processing, co precipitation in a non solvent or by tray drying method.

21. A composition as claimed in claim 1 wherein the taste masked drug polymer matrix in particulate form is suspended using the reconstitution medium of pH 4.5 comprising of sucrose, tutti-frutti flavor, citric acid and polyvinyl pyrrolidone.

22. A process for the preparation of a taste masked pharmaceutical composition comprising a pH sensitive polymer and a bitter drug, and of the formula P[A₍ₓ₎B_{(y)}C_{(z)}]:D wherein P is the pH sensitive polymer comprising (A) a hydrophobic monomer, (B) a basic monomer and (C) a hydrophilic monomer and (D) a bitter drug and (x)=30-95%, (y)=5-70%, (z)=0-60%, all expressed in terms of w/w and the ratio of (P) to (D) is in the range of 30:1 to 0.2:1 w/w, the process comprising dispersing or coating the drug in the form of microparticles within a matrix formed by the polymer.

23. A process as claimed in claim 22 wherein the hydrophobic monomer (A) is a acrylic or a methacrylic acid ester selected from the group consisting of cyclohexyl acrylate, dodecyl acrylate, 2 ethyl hexyl acrylate, octyl acrylate, tertiary butyl acrylate, phenyl acrylate, butyl acrylate, methyl methacrylate, benzyl methacrylate, cyclohexyl methacrylate, phenyl methacrylate, tertiary butyl methacrylate, butyl methacrylate, 2 ethyl hexyl methacrylate, propyl methacrylate preferably butyl acrylate, methyl methacrylate and butyl methacrylate.

24. A process as claimed in claim 22 wherein the basic monomer (B) is selected from the group consisting of amino alkyl acrylic acid and methacrylic acid esters selected from the group consisting of dimethyl amino ethyl methacrylate, dimethyl amino ethyl acrylate, diethyl amino ethyl methacrylate, diethyl amino ethyl acrylate, piperidine ethyl methacrylate, 2 tert-butyl amino ethyl methacrylate, preferably dimethyl amino ethyl methacrylate and diethyl amino ethyl acrylate

25. A process as claimed in claim 22 wherein the basic monomer (B) is an alkenyl pyridine selected from the group consisting of 2-vinyl pyridine, 3-vinyl pyridine, 4-vinyl pyridine and 5-vinyl 2 picoline, 2-vinyl 4 picoline, 2 isopropenyl pyridine, iso propenyl pyridine, preferably 4-vinyl pyridine.

26. A process as claimed in claim 22 wherein the basic monomer (B) is selected from vinyl quinolines, aminoalkyl vinyl ethers, amino ethyl styrenes and allylic amines, preferably allylic amines.

27. A process as claimed in claim 22 wherein the In yet another embodiment of the invention the hydrophilic monomer (C) is an acrylic or methacrylic acid ester selected from the group consisting of hydroxy ethyl methacrylate, hydroxy propyl methacrylate, hydroxy ethyl ethyl methacrylate, hydroxy ethyl acrylate, hydroxy propyl acrylate, hydroxy ethyl ethyl acrylate preferably hydroxy ethyl methacrylate and hydroxy ethyl ethyl methacrylate.

28. A process as claimed in claim 22 wherein the drug comprises a macrolide antibiotic selected from the group consisting of erythromycin, azithromycin and clarithromycin, fluroquinolones selected from the group consisting of ciprofloxacin, enrofloxacin, ofloxacin, gatifloxacin, levofloxacin and norfloxacin, cephalosporins selected from the group consisting of cefuroxime, cephalexin, cephadroxil, cepfodoxime proxetil, nonsteoroidal, and anti-inflammatory and analgesic drugs selected from the group consisting of ibuprofen and diclofenac sodium and COX 2 inhibitors selected from the group consisting of etoricoxib and celecoxib, antihistamic drugs selected from the group consisting of chlorpheniramine maleate, oxazolidinones selected from the group consisting of linezolid and other drug like dextromethorphan.

29. A process as claimed in claim 22 wherein the drug itself or its pharmaceutically acceptable salt or ester or amide is used.

30. A process as claimed in claim 22 wherein the total polymer to drug ratio is in the range of 30:1 to 0.2:1 by weight.

31. A process as claimed in claim 30 wherein the total polymer to drug ratio is in the range of 5:1 to 0.4:1 by weight.

32. A process as claimed in claim 22 wherein the pH sensitive polymer solubilizes or swells in the acidic pH <3 as found in stomach and remains insoluble or de swelled in the pH >3.5.

33. A process as claimed in claim 22 wherein the composition is prepared by microencapsulation using an emulsification solvent extraction method comprising dissolving the pH sensitive polymer in an organic solvent selected from acetone, methanol, dichloromethane and a mixture of methanol and dichloromethane in the ratio 1:1 to 1:1.5, adding the drug to the polymer solution to obtain a solution or a homogeneous dispersion, adding this organic phase to light liquid paraffin-containing span 85 in an amount of 0.1 to 1% w/w, continuously stirring the mixture mechanically at a rate of about 500 rpm and at a temperature of about 25° C. for a period of about 30 minutes, adding 40 ml of n-hexane or cyclohexane at the rate of 5 ml/min, followed by adding another 40 ml of n-hexane or cyclohexane rapidly, maintaining agitation for a period in the range of 10-15 min and then separating the microparticles by filtration and washing the separated microparticles with petroleum ether or n hexane and drying at a temperature of about 27° C. under vacuum for up to 24 hours.

34. A process as claimed in claim 22 wherein the polymer coated drug microparticles are obtained by spray drying comprising spraying the drug-polymer solution or dispersion in an organic solvent to obtain the taste masked micro particles, subjecting the sprayed microparticles to drying in the presence of a drying gas selected from the group consisting of nitrogen, argon, carbon dioxide and air.

35. A process as claimed in claim 34 wherein the gas inlet temperature to the spray dryer is dependent on the solvent used and is in the range of 35-150° C.

36. A process as claimed in claim 35 wherein the gas inlet temperature is in the range of 40-60° C.

37. A process as claimed in claim 34 wherein the gas outlet temperature is dependant on the solvent and is in the range of 25 to 50° C.

38. A process as claimed in claim 37 wherein the gas outlet temperature is in the range of 25 to 40° C.

39. A process as claimed in claim 34 wherein the polymer is solubilized in methanol or a mixture of methanol and dichloromethane (1:1) and the drug is either solubilized or dispersed in the polymer solution.

40. A process as claimed in claim 22 wherein the taste masked microparticles of the drug is mixed with a flavoring agent, citric and tartaric acids, sweeteners selected from sucrose, saccharin and aspartame, and other pharmaceutically acceptable excipients to be formulated as conventional, chewable or dispersible tablets, dry syrups, suspensions, sachets or any other suitable oral dosage form.
